(19) United States
(12) Patent Application Publication
(10) Pub. No.: US 2020/0381090 A1
(43) Pub. Date: Dec. 3, 2020

(54) Patient Context Vectors: Low Dimensional Representation of Patient Context Towards Enhanced Rule Engine Semantics and Machine Learning

(71) Applicant: Computer Technology Associates, Inc., Cardiff, CA (US)

(72) Inventors: Emilia Apostolova, Chicago, IL (US); Carmelo Velez, Encinitas, CA (US); Timothy Tschampel, Ashburn, VA (US)

(73) Assignee: Computer Technology Associates, Inc., Cardiff, CA (US)

(21) Appl. No.: 16/888,199

(22) Filed: May 29, 2020

Related U.S. Application Data
(60) Provisional application No. 62/854,256, filed on May 29, 2019.

(51) Int. Cl. G16H 10/60; G16H 50/30; G16H 50/20; G06N 20/00

(52) U.S. Cl. CPC G16H 10/60; G06N 20/00; G16H 50/20; G16H 50/30

(57) ABSTRACT

A PCV generation process using deep learning networks and multi-task learning wherein what knowledge is already known can be used to learn new knowledge such as the addition of CPT and medication information to augment patient PCVs based on ICD codes and expressions of history in free text notes.

### CROSS REFERENCE TO RELATED APPLICATIONS

The present application claims the benefit of provisional 62/854,256, filed on May 29, 2019, the entirety of which is incorporated by reference.

### TECHNICAL FIELD

The present disclosure is generally directed towards methods and systems for rule engines and training machines to categorize data, and/or recognize patterns in data, and to machines and systems relating thereto. More specifically, exemplary aspects of, the invention relate to methods and systems for deriving features that include low dimensional representation of patient context to create enhanced rule engine semantics and machine learning.

### BACKGROUND OF THE DISCLOSURE

Automated detection and prediction of high risk in hospitalized patients plays a pivotal role in modern healthcare informatics, with the goals of early recognition, treatment and prevention of life-threatening diseases. Recently, rule engines and machine learning (ML) have emerged as methods of implementing disease detection and prediction in bedside clinical decision support systems. Rule engines (used as electronic medical records (EMR) data screening tools to detect disease from non-specific signs or symptoms) frequently use risk factors extracted from EMR data elements that have shown to be associated with a disease outcome. The number of potential risk factor variables in a typical patient the electronic health record (EHR) may easily number in the thousands, particularly if free-text notes from doctors, nurses, and other providers are included. For practical reasons, many of the current rule-based screening tools are “parsimonious”, relying on a few selected features to minimize redundancies and maximize utility. Similarly, the predictive performance of current ML classification algorithms trained using electronic medical record (EMR) data relies heavily on adequate selection of features that contribute to class separability while achieving dimensionality reduction in which irrelevant, weakly relevant or redundant features are detected and removed.

Dimensionality reduction also plays an important direct role in ML classification performance [1]. The features needed for a reliable risk evaluation of a variety of patient conditions must be extracted from high volume, redundant data typically dispersed across the patient EMR, and available at different times throughout the patient stay. The patient demographics, past medical and visit history, chronic conditions, risk factors, current signs and symptoms can be found in the form of clinical notes (e.g. nursing notes, radiology reports, etc.), diagnosis and procedure codes, vital signs, lab orders and results. Thus, a major challenge of EMR-based screening tools and machine learning is the combining and selection of optimal feature sets from this variability and volume of EMR data, resulting from different charting behaviors, health care delivery models, hospital settings, etc.

For example, current disease-focused rule-based screening tools and ML efforts for acute syndromic diseases such as sepsis or acute respiratory disease syndrome (ARDS) generally rely on features determined relevant in observational studies and, more importantly, expert consensus-based medical criteria. For example, sepsis, currently defined as a life-threatening organ dysfunction caused by a dysregulated host response to an infection [2] is associated with infection-induced organ dysfunction indicated by abnormal vital signs and lab results. Similarly, ARDS is a life-threatening respiratory condition characterized by acute onset of hypoxemia triggered by number of inciting insults to the lungs including trauma, sepsis, aspiration, etc. indicated by abnormal blood oxygenation measurements and lung damage seen in chest radiology examinations [3]. The early recognition of these rapidly progressive conditions and/or the identification of those at high risk can save lives. However, the initial signs and symptoms of syndromes such as sepsis and ARDS are frequently nonspecific (e.g. abnormal vitals and labs with variable etiologies), commonly involving confounding complex interactions of large numbers of multiple patient-specific risk factors, comorbidities and current signs/symptoms, frequently leading to misdiagnosis and/or delays in manually derived diagnosis by bedside clinicians. Thus, what is needed are rule-based EMR data surveillance screening tools and predictive models that comprehensively capture the high-volume myriad class-defining patient-specific conditions to assist in early recognition and treatment of these critical conditions.

For effective rule-based screening and predictive analytics, in addition to acute features such as vitals and labs, patient medical “context” in the form of predisposing risk factors such as those patients with a compromised immune system (e.g. patients with cancer, HIV, diabetes, recent surgeries, etc.) are also considered important features. In many elderly patients pre-disposing context may involve numerous co-morbidities (e.g. represented as an ICD problem list in the patient EMR) that may result in high risk interactions that should be represented as features. Intuitively, the totality of patient history captured in a problem list comprised as a set of patient's diagnosis codes can represent a meaningful medical summary of the patient. In current electronic medical records, diagnosis codes are used to describe both current diagnoses (e.g. a patient presenting with community-acquired pneumonia), but also a variety of additional facts. For example, ICD codes can describe patient's history and chronic conditions (e.g. Chronic kidney disease; Personal history of traumatic fracture; etc.); information regarding past and current treatments and procedures/interventions (e.g. Infection due to other bariatric procedure, mental health tests/psychotherapy, surgeries, radiation therapy, etc.). In some cases, ICD codes contain information such as the patient age group (e.g. Sepsis of newborn; Elderly multigravida); expected outcome (Encounter for palliative care); patient's social history (e.g. Adult emotional/psychological abuse); the reason for the visit, (e.g. railway/motor vehicle accidents, near drowning, respiratory distress, etc.).

While there are many ICD codes, they tend to be interdependent, and to co-occur. For example, Pneumonia ICD codes are often accompanied with ICD codes describing Cough, Fever, Pleural effusion, etc. Inspired by word embeddings [6], it has been suggested that this medical code co-occurrence can be exploited to generate low dimensional representations of ICD codes.

Given that there are nearly 70,000 ICD codes the identification and representation of complex combinations of this contextual knowledge for use in disease-specific rule engines and in ML training is dimensionally challenging.

More importantly, patient context information might be present only in the form of free-text notes, and not available in the form of ICD codes. Creating suitable low-dimensional representation of clinical free-text, that can be easily combined with EMR structured data, remains a challenge.

Thus, there exists a need in the art to address the problems described above.

### SUMMARY OF THE DISCLOSURE

Aspects the present invention meet the above-identified unmet needs of the art, as well as others, by providing tools and methods and systems for recognizing patterns in complex data. The present disclosure involves converting low dimensional representations of clinical knowledge to ontology-guided rule engines. It can be appreciated that this can automatically extend the knowledgebase by data-driven discovery of disease patterns, such as comorbidities, predisposing risk factors, patient phenotype-specific treatment outcomes, etc. When used in combination with new clinical findings, this method can detect the likely presence of a disease or be used as predisposing risk factor features for ML-based predictions of impending patient deterioration enabling preventive measures that can improve outcomes.

Although specific advantages have been enumerated above, various embodiments may include some, none, or all of the enumerated advantages. Additionally, other technical advantages may become readily apparent to one of ordinary skill in the art after review of the following figures and description.

### BRIEF DESCRIPTION OF THE DRAWINGS

For a more complete understanding of the present disclosure and its advantages, reference is now made to the following description taken in conjunction with the accompanying drawings, in which like reference numerals represent like parts.

FIG. 1 discloses a Real-time ARDS prediction workflow using patient context vectors.

FIG. 2 discloses a method to generate patient context vectors from ICD codes and free text patient descriptions

### DETAILED DESCRIPTION OF THE DISCLOSURE

It should be understood at the outset that, although exemplary embodiments (ARDS prediction) are illustrated in the figures and described below, the principles of the present disclosure may be implemented in support of automated detection and prediction of other life threatening diseases using other rule engine or machine learning techniques. The present disclosure should in no way be explicitly limited to the exemplary implementations and techniques illustrated in the drawings and described below. Additionally, unless otherwise specifically noted, articles depicted in the drawings are not necessarily drawn to scale

The methods disclosed in the present invention include generating a “Patient Context Vector” (PCV). PCV is a data structure that is a low-dimensional representation of a patient's medical context (history and present condition) obtained in self-supervised manner by utilizing historical EMR data. It can be appreciated that a PCV is thus an embeddings of multi-dimensional patient data (diagnosis, procedure codes, clinical texts, etc.) to a continuous vector space with much lower dimension. PCVs utilize available EMR patient information (such as a patient's history, current symptoms and conditions) for low dimensional contextual predictive modelling, including real-time predictions. The described method is applicable to a variety of use cases needing summarized high volume information dispersed across the EMR patient record.

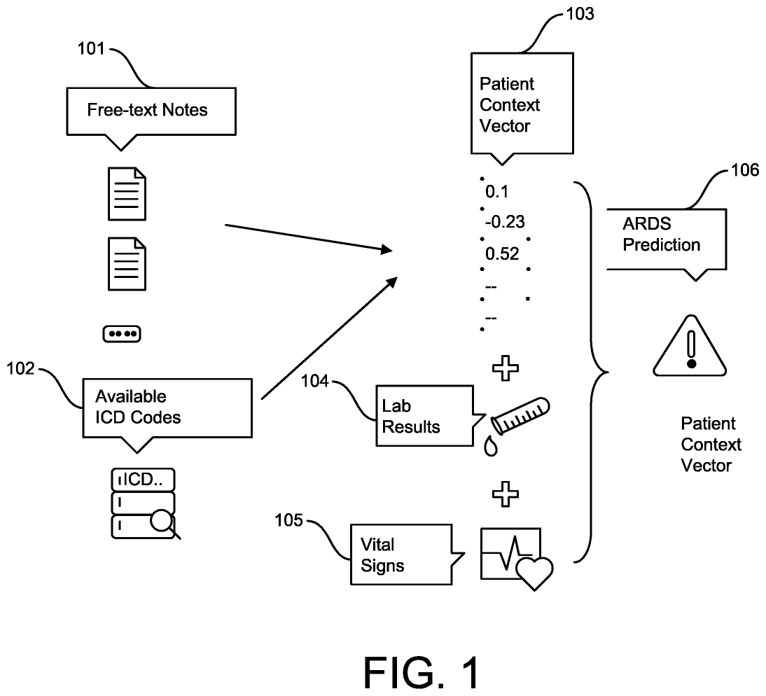

FIG. 1 discloses a Real-time ARDS prediction 106 workflow. Nursing notes 101 available at prediction time are used to predict Patient Context Vectors 103. ICD codes 102 available at prediction time are also converted to Patient Context Vectors 103 by averaging ICD code embeddings. Patient Context Vectors are used together with structured EMR data (lab results 104 and vital signs 105) to predict ARDS status.

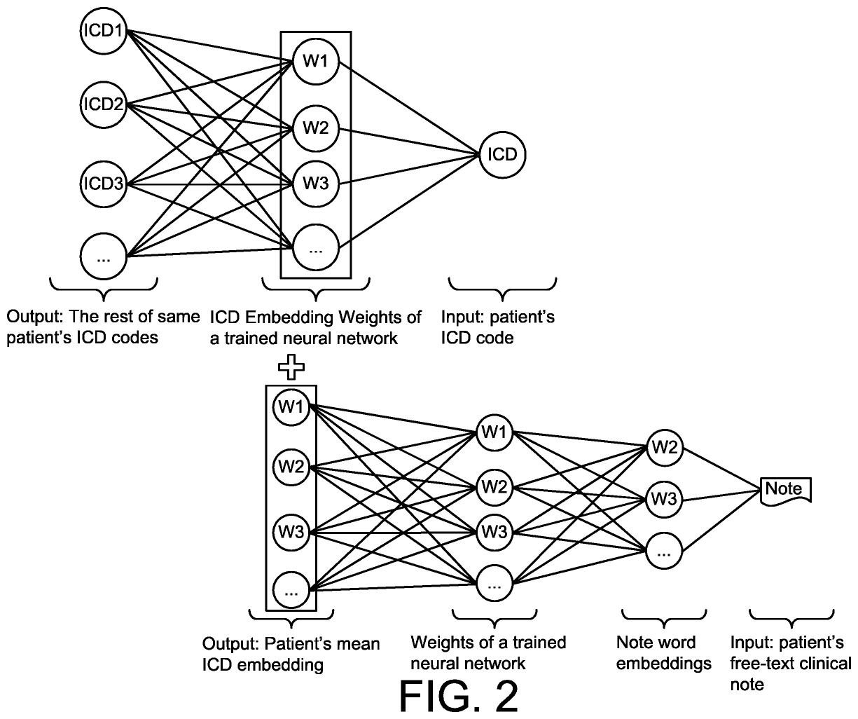

At prediction time, PCVs are generated from the combination of available up-to-date ICD codes (if any) and available clinical notes. In FIG. 2, a deep learning network is trained on all available data, that, given a patient's ICD code (network input) predicts the rest of the patient's ICD codes (network output). The weights of the trained network (shown inside a red rectangle) represent the ICD embedding. Each of the patient's ICD codes is thus mapped to fixed-size vector embeddings, which are then averaged. A second deep neural network (e.g. Convolutional Neural Network or Transformer network) is then trained to predict the patient's averaged ICD embeddings from the patient's free-text notes. At prediction time, each of the available patient's ICD codes, and clinical notes are converted to ICD embeddings (red boxes) and averaged, representing the Patient's Context Vector. Similar approach can be taken to additional multi-dimensional EMR structured data, such as CPT codes and medication lists. Once CPT code embeddings and medication embeddings are generated, a deep learning network can be trained to jointly predict patient's ICD, CPT, medication embeddings from free-text notes via multi-task learning. In one embodiment, PCVs (vectors of real numbers) can be simply added to the list of existing structured data variables (vital signs and lab results) and used in a variety of rule engine and machine learning models. Predictive models can be used for a variety of applications such as 1) identifying patients at risk of developing life-threatening conditions 2) identifying patient cohorts, and 3) clustering to determine phenotypes of specific conditions for targeted personalized treatments, etc.

In a further embodiment, low-dimensional representation of ICD codes (ICD embeddings) are generated from a large corpus of patient ICD records. All unique codes in the corpus are converted to ICD embeddings (vectors of real numbers). The embeddings are created by using all patient data in an unsupervised neural network, i.e. given a patient's code X, predict the rest of their codes, or alternatively, given a list of codes, predict what other codes a patient has. Patient visit EMR data is used to look up recorded up-to-date ICD codes, clinical notes, vital signs, and lab results. The visit ICD codes are converted to embeddings and averaged to produce Patient Context Vectors. For example, by experimenting, for ARDS, the optimal vector dimension was determined to be of size 50.

To support predictive analytics wherein complete problem lists may not be available in real-time, a deep learning model is trained to predict the patient's Patient Context Vector from clinical notes (e.g. early encounter nursing and physician notes). The Patient Context Vectors obtained from available EMR ICD codes, and from free-text notes are then used in conjunction with vital signs, and lab results to predict the patient's outcome.

Modifications, additions, or omissions may be made to the systems, apparatuses, and/or methods described herein without departing from the scope of the disclosure. For example, various components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

1. A method comprising:
importing, by a processor, free text notes and available ICD codes;
generating a “patient context vector” (PCV) from the free text notes and available ICD condes, wherein the PCV is a low-dimensional representation of a disease-specific contextual knowledge, wherein the PCV includes what physicians know about a patient apart from clinical signs and symptoms;
combining the patient context vector with patient EMR data to predict life threatening disease status.

2. The method of claim 1 further comprising using a deep learning network to learn new knowledge including the addition of CPT and medication information to augment patient PCVs based on ICD codes and expressions of history in free text notes.

3. A machine learning method comprising:
generating a plurality of PCVs from the combination of available up-to-date ICD codes and available clinical notes utilizing historical EMR data in an unsupervised manner PCVs are low-dimensional representations of patient's medical history and present condition;
adding the generated PCVs to a plurality of existing structured data variables, wherein the plurality of existing structured data variables further include vital signs and lab results; and
identifying patients at risk of developing life-threatening conditions.
